(19) United States
(12) Patent Application Publication
(10) Pub. No.: US 2021/0282643 A1
(43) Pub. Date: Sep. 16, 2021

(54) System And Method For Detecting Tau Protein In Ocular Tissue

(71) Applicant: Cognoptix, Inc., Concord, MA (US)

(72) Inventors: Daniel R. Vlock, Cambridge, MA (US); Joyce Myers, Cambridge, MA (US); Dennis J. Nilan, Milford, MA (US); Charles Kerbage, Arlington, MA (US)

(21) Appl. No.: 16/321,683

(22) PCT Filed: Jul. 31, 2017
(86) PCT No.: PCT/US2017/044592

§ 371 Date: Jan. 29, 2019

Related U.S. Application Data
(60) Provisional application No. 62/369,570, filed on Aug. 1, 2016.

(51) Int. Cl. A61B 5/00; A61B 3/14

(52) U.S. Cl. CPC A61B 5/0071; A61B 3/14; A61B 5/0082; A61B 5/4082; A61B 5/4088

(57) ABSTRACT

A system and method for detecting in an eye, in particular the retina, of a mammal, tau protein, such as neurofibrillary tangles (NFT). The system and method can use fluorescence imaging in the visible or near-infrared spectral region, for example as a non-invasive method for in vivo imaging of tau protein in ocular tissue. In preferred embodiments, in vivo imaging of tau protein in ocular tissue involves tau-binding compounds such as FDDNP, T807 and T808.

### RELATED APPLICATIONS

This application claims the benefit of U.S. Provisional Application No. 62/369,570, filed on Aug. 1, 2016. The entire teachings of the above application are incorporated herein by reference.

### BACKGROUND

The retina is part of the central nervous system, with various cell types, including photoreceptors, bipolar cells, horizontal cells, amacrine cells and retinal ganglion cells. In a study based on immunohisto-chemical analysis of 19 nucleated eyes from patients aged 49-87 years, diffuse immunoreactivity of tau was found in the inner nuclear layer in all patients, while it was found in seven cases in retinal ganglion cells. (1)

Several studies suggest that the visual system is affected by neurodegenerative processes in probable Alzheimer's Disease (AD) patients. Several ocular imaging techniques demonstrate that pathological changes within the retina could be found in such patients. Optical coherent tomography has demonstrated a significant reduction of peri-papillary retinal fiber layer thickness (RFL) in patients with early AD when compared with age-matched controls. Changes in the optic nerve head have also been observed, using confocal scanning laser ophthalmoscopy. The observed changes included reduced RFL thickness, neuroretinal rim volume and area, and an increased cup-disc ratio, suggesting an overall reduction in the number of optic nerve fibers passing through the optic nerve head. Moreover, laser Doppler flowmetry has demonstrated that retinal blood-flow rate is reduced in AD patients.

There is, however, an ongoing need for techniques of detecting and measuring tau protein in ocular tissue with high specificity and sensitivity.

### SUMMARY OF THE INVENTION

In accordance with an embodiment of the invention, there is provided a method for detecting in an eye, in particular the retina, of a mammal, tau protein, such as neurofibrillary tangles (NFT). The method can use fluorescence imaging in the visible or near-infrared spectral region, for example as a noninvasive method for in vivo imaging of tau protein in ocular tissue.

In one embodiment according to the invention, there is provided a method of optical detection of a tau protein in ocular tissue. The method comprises illuminating the ocular tissue with a light source, the light source having an excitation wavelength appropriate to produce fluorescence in at least a tau-binding compound when bound to the tau protein in the ocular tissue, the tau-binding compound having been introduced to the ocular tissue and specifically binding to the tau protein. The method further comprises detecting at least a portion of fluorescence, emitted from the ocular tissue by the tau-binding compound bound to the tau protein, upon illumination of the ocular tissue with the light source, the emitted fluorescence having an emission wavelength in the range of from about 550 nm to about 1400 nm, such as from about 750 nm to about 1400 nm.

In further, related embodiments, the excitation wavelength may be in the range of from about 550 nm to about 1400 nm, such as from about 750 nm to about 1400 nm. The method may comprise determining at least one of an intensity and a time decay rate of the at least a portion of fluorescence emitted from the ocular tissue by the tau-binding compound bound to the tau protein; and determining a quantity of the tau-binding compound bound to the tau protein based on at least one of the intensity and the time decay rate. The method may further comprise detecting at least one of: (i) at least a portion of background autofluorescence emitted from a corresponding portion of the ocular tissue, upon illumination of the ocular tissue with the light source, and (ii) at least a portion of fluorescence emitted from the ocular tissue by a quantity of the tau-binding compound that is unbound to the tau protein; and normalizing the determined quantity of the tau-binding compound bound to the tau protein based on at least one of: (i) the at least a portion of the background autofluorescence emitted from the corresponding portion of the ocular tissue and (ii) the at least a portion of the fluorescence emitted by the quantity of the unbound tau-binding compound. The method may further comprise performing a time-correlation single photon counting of the at least a portion of fluorescence emitted from the ocular tissue by the tau-binding compound bound to the tau protein.

In other, related embodiments, the emission wavelength may differ from the excitation wavelength by a wavelength shift towards an increased wavelength, for example a shift of between about 10 nm and about 100 nm. The tau-binding compound may produce a significantly greater intensity of emitted fluorescence when exposed to the excitation wavelength and bound to the tau protein, as compared with intensity of emitted fluorescence by the tau-binding compound when exposed to the excitation wavelength and unbound to the tau protein. For example, the significantly greater intensity of emitted fluorescence may comprise at least one of at least five times greater intensity of emitted fluorescence, at least ten times greater intensity of emitted fluorescence, and at least twenty times greater intensity of emitted fluorescence.

In further related embodiments, the tau-binding compound may comprises a terminal electron-rich donor group; a terminal electron-deficient acceptor group; and a polarizable pi-conjugated system bridging the terminal electron-rich donor group and the terminal electron-deficient acceptor group. The tau-binding compound may be a compound with the following structure:

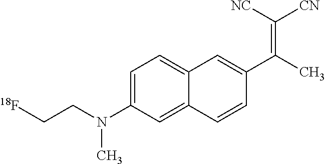 [SMILES: CC(=C(C#N)C#N)c1ccc2cc(N(C)CC[18F])ccc2c1]

or a pharmaceutically acceptable salt thereof.

The tau-binding compound may be a compound with the following structure:

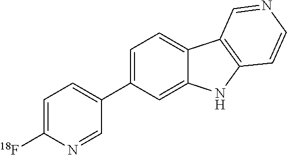 [SMILES: [18F]c1ccc(-c2ccc3c(c2)[nH]c2ccncc23)cn1]

or a pharmaceutically acceptable salt thereof.

The tau-binding compound may be a compound with the following structure:

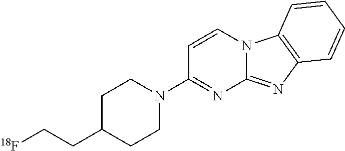 [SMILES: [18F]CCC1CCN(c2ccn3c(n2)nc2ccccc23)CC1]

or a pharmaceutically acceptable salt thereof.

In other related embodiments, the tau protein may comprise at least one of: a plurality of paired helical Tau-filaments; a neurofibrillary tangle; a Tau protein aggregate precursor; and a Tau protein aggregate. The ocular tissue may comprise at least a portion of a retina of an eye, such as at least one of an inner nuclear layer of the retina, and a retinal ganglion cell of the retina. The detecting the at least a portion of the fluorescence, emitted from the ocular tissue by the tau-binding compound bound to the tau protein, may be used for aiding in diagnosis of disease, such as an amyloidogenic disease. The diseases may be selected from the group consisting of Alzheimer's disease (AD), familial AD, Sporadic AD, Creutzfeld-Jakob disease, variant Creutzfeld-Jakob disease, spongiform encephalopathies, Prion diseases (including scrapie, bovine spongiform encephalopathy, and other veterinary prionopathies), Parkinson's disease, Huntington's disease (and trinucleotide repeat diseases), amyotrophic lateral sclerosis, Down's Syndrome (Trisomy 21), Pick's Disease (Frontotemporal Dementia), Lewy Body Disease, neurodegeneration with brain iron accumulation (Hallervorden-Spatz Disease), synucleinopathies (including Parkinson's disease, multiple system atrophy, dementia with Lewy Bodies, and others), neuronal intranuclear inclusion disease, tauopathies (including progressive supranuclear palsy, Pick's disease, corticobasal degeneration, hereditary frontotemporal dementia (with or without Parkinsonism), a pre-morbid neurodegenerative state and Guam amyotrophic lateral sclerosis/parkinsonism dementia complex). The method may comprise aligning the light source with at least a portion of the ocular tissue, such as by forming a camera image of a retina to be illuminated with the light source.

In another embodiment according to the invention, there is provided a device for optical detection of a tau protein in ocular tissue. The device comprises a light source configured to emit light to illuminate the ocular tissue, the light source having an excitation wavelength appropriate to produce fluorescence in at least a tau-binding compound when bound to the tau protein in the ocular tissue, the tau-binding compound having been introduced to the ocular tissue and specifically binding to the tau protein. An optical unit is configured to detect at least a portion of fluorescence, emitted from the ocular tissue by the tau-binding compound bound to the tau protein, upon illumination of the ocular tissue with the light source, the emitted fluorescence having an emission wavelength in the range of from about 550 nm to about 1400 nm, such as from about 750 nm to about 1400 nm. In further, related embodiments, the light source and the optical unit may be configured to implement any of the methods taught herein.

In further, related embodiments, the optical unit may further comprise a time decay calculation module. The optical unit may comprise a time correlation single photon count module configured to perform a time-correlation single photon counting of the at least a portion of fluorescence emitted from the ocular tissue by the tau-binding compound bound to the tau protein. The device may further comprise a camera configured to form a camera image of a retina to be illuminated with the light source.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic block diagram of a method of optical detection of a tau protein in ocular tissue, in accordance with an embodiment of the invention.

FIG. 2 is a schematic diagram of an optical device in accordance with an embodiment of the invention.

### DETAILED DESCRIPTION OF THE INVENTION

A description of example embodiments of the invention follows.

In accordance with an embodiment of the invention, there is provided a method for detecting in an eye, in particular the retina, of a mammal, tau protein, such as neurofibrillary tangles (NFT).

In accordance with an embodiment of the invention, fluorescence imaging in the visible or near-infrared (NIR) spectral region, such as in the region between about 550 nm to about 1400 nm wavelength, for example in the region between about 750 nm to about 1400 nm of excitation wavelength, emission wavelength or both, is used as a noninvasive method for in vivo imaging of tau protein in ocular tissue, such as the retina. In the NIR region, biomolecules have low absorption and autofluorescence, thus allowing an optimal penetration depth and high sensitivity. In particular, the NIR fluorescence labeling of NFT is of special interest because accumulated evidence has suggested that the severity of dementia correlates better with the load of tau fibrils than with amyloid-beta (Aβ). Tau-targeting probes have emerged more slowly than amyloid probes, and only a handful of chemical entities that function as molecular probes for NFT or tau aggregates have been identified. In terms of imaging probes for tau pathology, the examples are limited.

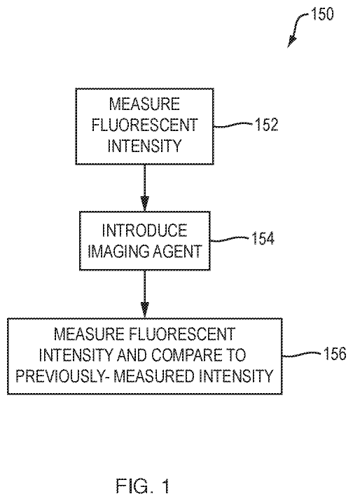

FIG. 1 is a schematic block diagram of a method of optical detection of a tau protein in ocular tissue, in accordance with an embodiment of the invention. In the embodiment of FIG. 1, a method of optical detection of a tau protein in ocular tissue includes the stages shown. The process 150, however, is exemplary only and not limiting. The process 150 may be modified, e.g., by adding, removing or rearranging stages. For example, stage 152 may be removed and stage 156 modified to eliminate comparing measured intensity with previously-measured intensity.

At stage 152, ocular tissue—for example, the retina—is illuminated and fluorescence measured. The ocular tissue is illuminated with a light source and fluorescence emitted from the eye in response to the illumination measured and recorded. The magnitudes of emitted fluorescence and the locations of these magnitudes are correlated and recorded.

At stage 154, an imaging agent is introduced into the ocular tissue, for example, the retina. The imaging agent is configured to bind to materials/objects of interest, such as tau protein, and is configured to fluoresce in response to light from the source. The imaging agent may be introduced in a variety of manners, e.g., through drops applied to the eye, intravenously, etc. The imaging agent can, for example, include any of the tau-binding compounds taught herein.

At stage 156, the eye, such as the retina, is illuminated with light from the source and the fluorescence from the eye measured. The intensity magnitudes and locations are correlated and stored, and compared with magnitudes recorded at stage 152, with magnitudes measured from similar locations in stages 152 and 156 being compared. The comparison includes analyzing differences in the magnitudes and determining presence of the material/object of interest, such as tau protein, and the amount of the material/object (such as tau protein) to the extent present in the eye. Conclusions can be determined regarding implications of the presence and/or amount of the material/object of interest (such as tau protein), such as a medical condition of the subject, such as the existence and/or stage of a disease such as Alzheimer Disease.

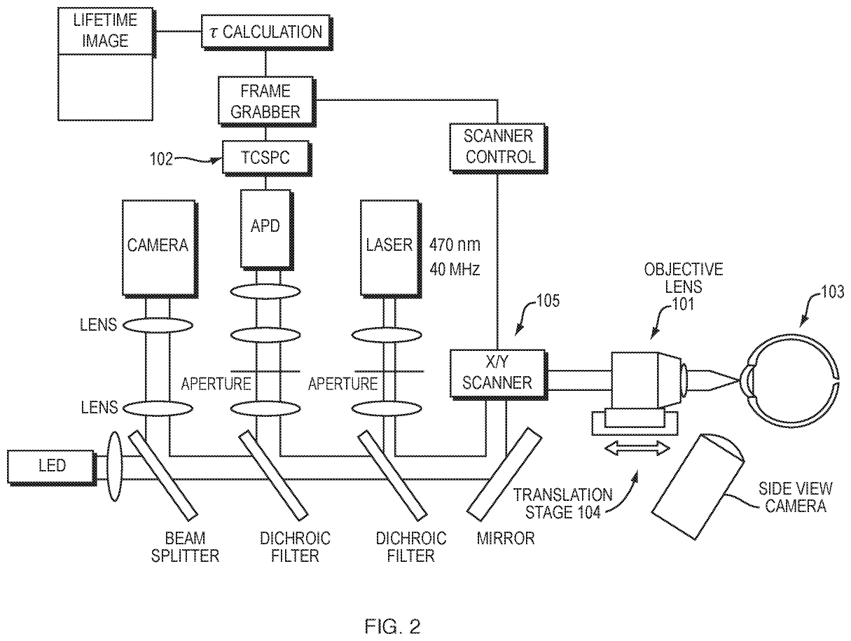

FIG. 2 is a schematic diagram of an optical device in accordance with an embodiment of the invention. In this embodiment, a time correlation single photon counting (TCSPC) technique is used to detect and measure fluorescence emitted from the retina. However, it will be appreciated that other fluorescence imaging techniques can be used, including those that do not use fluorescence lifetimes and that rely on fluorescence intensity only.

In the embodiment of FIG. 2, fluorescence excitation is achieved by a pulsed laser beam that is focused by a high numerical aperture objective lens 101 into the eye. Fluorescence is detected using a time correlation single photon counting (TCSPC) technique through a confocal configuration with a fast avalanche photodiode detector (APD) 102. TCSPC is performed by using a short pulse of light to excite the sample (eye) 103 repetitively, and recording the subsequent fluorescence emission as a function of time. This usually occurs on the nanosecond timescale. The ocular tissue examined may, for example, be the retina.

In the embodiment of FIG. 2, identification of the anatomical structures of the ocular tissue, such as the retina, is performed by scanning the objective lens 101 on axis using a translation stage 104. The signal is measured at every point along the scan in order to reveal the anatomical structures of the ocular tissue, such as the retina. In addition, the scan provides information about the pharmacokinetics of exogenous tau-binding compounds applied to the eye. Such information provides not only spatial and temporal information of the tau-binding compound, but also the concentration of the tau-binding compound in the ocular tissue, such as the retina.

In another embodiment, identification of the anatomical structures of the ocular tissue, and of the location of interest, can be performed by merely obtaining a general camera image of the retina, without performing a scan along an axis.

In the embodiment of FIG. 2, once the location of interest in the ocular tissue—such as the location of interest in the retina—is known from the excited natural fluorescence measured at every point along the axial scan, another scan is executed in a plane (xy) perpendicular to the optical axis using a set of galvanometer mirrors 105. To ensure allocation of the measured fluorescence decay curves to the corresponding site of the two-dimensional scanning, the galvanometer set scanning is synchronized with the laser pulses and photodetection for time-correlated individual photon counting. In the embodiment of FIG. 2, one or more modules may be implemented using dedicated, specialized hardware modules and/or using a general purpose computer specially-programmed to perform the modules' functionality, including, for example, the Frame Grabber module, TCSPC module, τ Calculation module and scanner control module. A general purpose computer and/or one or more specialized hardware modules may receive data from each other via data cables and data ports appropriate for the modules' functionality.

In the embodiment of FIG. 2, for time-correlated individual photon counting, the decay curve of the autofluorescence is registered for each scanned location of the ocular tissue, such as the retina, and thus a two-dimensional representation of the fluorophores' distributions can be evaluated and analyzed based on their fluorescence decay time as well as on their intensity. The image of the calculated decay times can be encoded by false colors and can be superimposed on the intensity image for better clinical interpretation. Since the fluorescence decay time is a characteristic for each fluorescence molecule, one can determine and separate the fluorophores (for example, tau-binding compound bound to tau protein, from natural fluorescence of the ocular tissue, such as the retina, and from tau-binding compound that is unbound to tau protein) being excited in the sample volume. By combining fluorescence intensity and lifetime measurements, an extra dimension of information can be obtained to discriminate among several fluorescent labels.

In accordance with an embodiment of the invention where a TCSPC technique (for example, using the device of FIG. 2, above), is used, the method can comprise illuminating the eye with a light source having at least one of a wavelength property and a polarization property appropriate to produce fluorescence in at least an tau-binding compound when the tau-binding compound is bound to the tau protein, the tau-binding compound having been introduced to the eye and specifically binding to the tau protein indicative of the neurodegenerative disorder; and determining a time decay rate of fluorescence for at least the fluorescence produced by the tau-binding compound bound to the tau protein, the determining permitting distinguishing of the presence of the tau-binding compound bound to the tau protein in the eye based on at least the time decay rate.

In further, related embodiments, the method may further comprise determining a peak intensity of fluorescence for at least the fluorescence produced by the tau-binding compound bound to the tau protein. A quantity of the tau-binding compound bound to the tau protein may be determined, based on at least one of the peak intensity and the time decay rate. The method may further comprise determining a location of an ocular interface such as an interface of the retina of the eye based on an increase in a fluorescent signal due to natural fluorescence emitted from tissues of the eye. At least one region of the eye may be sampled using illumination by the light source, the sampling comprising performing at least one of a measure of the entire region or a sampling of different locations within the region or regions using illumination by the light source, the sampling of different locations comprising illuminating at least one point, plane and/or volume within the eye. The sampling may comprise sampling different locations across more than one region of the eye. The distinguishing the presence of the tau-binding compound bound to the tau protein may comprise distinguishing the tau-binding compound bound to the tau protein from background autofluorescence of other non-specific particles as well as unbound imaging agent. The method may comprise distinguishing at least one of a presence and a quantity of more than one of the following: the tau-binding compound; the tau-binding compound bound to the tau protein. The tau protein may comprise an aggregate or a pre-tau protein aggregate.

In accordance with an embodiment of the invention, the light source may be configured to emit light of an appropriate wavelength for a peak region of a fluorescent excitation spectrum for a fluorophore in the eye, such as in the retina; and an optical scanning system may be configured to detect light of an appropriate wavelength for a peak region of a fluorescent emission spectrum for the fluorophore (when bound to the tau protein, and/or when unbound to the tau protein) and/or the autofluorescence of the ocular tissue, such as the retina. For example, the excitation spectrum may have a peak between about 550 nm and about 1400 nm, such as between about 750 nm to about 1400 nm, the light source being configured to emit light within plus or minus about 10 nm to about 100 nm of the peak of the excitation spectrum, such as 20 nm above the peak of the excitation spectrum. The repetition rate of the pulsed laser can, for example, be from 30 to 70 MHz, such as from 40 to 60 MHz, for example from 45 to 55 MHz.

In accordance with an embodiment of the invention, the method can include the use of a molecular biomarker that can be utilized for fluorescence imaging of tau fibrils that have specific binding characteristics and can, for example, induce a redshift in the fluorescence emission. This can, for example, be achieved by employing terminal electron-rich donor and electron-deficient acceptor groups that are bridged by a highly polarizable π-conjugated system. In addition to redshifts, biomarkers can be selectively turned on when they bind to the target because the nonlinear optical properties that are associated with this architecture make the fluorescence intensity susceptible to the environmental changes that are conferred by target binding.

In one embodiment according to the invention, the tau-binding compound is a compound with the following structure, or a pharmaceutically acceptable salt thereof:

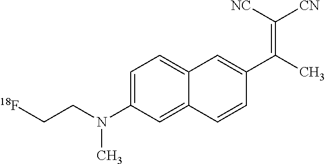 [SMILES: CC(=C(C#N)C#N)c1ccc2cc(N(C)CC[18F])ccc2c1]

Such a compound is known as [¹⁸F]-FDDNP, or 2-(1-{6-[(2-[¹⁸F]fluoroethyl)(methyl)amino]-2-naphthyl}ethyli-dene)malononitrile. The foregoing compound, [¹⁸F]-FDDNP, is a dialkylamino-naphthylethylidene derivative, and it will be appreciated that other dialkylamino-naphthylethylidene derivatives may be used as tau-binding compounds in accordance with an embodiment of the invention.

As used herein, the term “compound” also comprises pharmaceutically acceptable salts of the compounds as defined herein. The phrase “pharmaceutically acceptable salt(s)”, as used herein, refers to salts of compounds of the invention that are safe and effective for use in mammals and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. In one embodiment, a compound of the invention can form a pharmaceutically acceptable salt with an amino acid. Suitable base salts include, but are not limited to, aluminum, calcium, lithium, magnesium, potassium, sodium, zinc, and di-ethanolamine salts. In an embodiment, a pharmaceutically acceptable salt of a compound according to the invention is a hydrohalogenide salt, such as a hydrochloride or hydro-bromide salt, for example a hydrochloride salt.

In another embodiment according to the invention, the tau-binding compound is a compound with the following structure, or a pharmaceutically acceptable salt thereof:

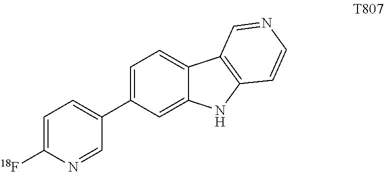 [SMILES: [18F]c1ccc(-c2ccc3c(c2)[nH]c2ccncc23)cn1]

Such a compound is known as [¹⁸F]T807, or (7-(6-fluoropyridin-3-yl)-5H-pyrido[4,3-b]indole). The foregoing compound, [¹⁸F]T807, is a benzimidazole pyrimidine derivative, and it will be appreciated that other benzimidazole pyrimidine derivatives may be used as tau-binding compounds in accordance with an embodiment of the invention.

In another embodiment according to the invention, the tau-binding compound is a compound with the following structure, or a pharmaceutically acceptable salt thereof:

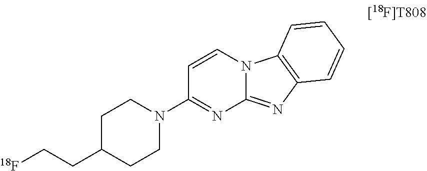 [SMILES: [18F]CCC1CCN(c2ccn3c(n2)nc2ccccc23)CC1]

Such a compound is known as [¹⁸F]T808, or 2-(1-(6-[(2-[¹⁸F]fluoroethyl)(methyl)amino]-2-naphtyl)ethylidene) malononitrile.

Although the above tau-binding compounds are shown as being radiolabeled (here, with an isotope of fluorine, ¹⁸F), embodiments according to the invention can also use modified versions of those compounds that are not radiolabeled, for example by removing the radioactive isotope or replacing it with a non-radioactive isotope, such as a non-radioactive fluorine atom or other non-radioactive group. For example, non-radioactive versions of [¹⁸F]-FDDNP, [¹⁸F]T807 and [¹⁸F]T808 can be used. It will be appreciated that other tau-binding compounds may be used, including non-radioactive tau-binding compounds, in accordance with an embodiment of the invention. Further, any of the tau-binding compounds taught herein can be chemically modified to change one or more of the excitation wavelength and emission wavelength of the tau-binding compound when bound or unbound to tau protein. For example, such tau-binding compounds can be chemically modified to optimize or improve performance for fluorescence imaging, including for visible or near infrared fluorescence imaging of such compounds. In one example, one or more of the tau-binding compounds taught herein is chemically modified such that one or more of its excitation wavelength and emission wavelength is in the visible or near infrared. In addition, more than one of the tau-binding compounds taught herein may be imaged simultaneously, in accordance with an embodiment of the invention.

In accordance with an embodiment of the invention, any of the tau-binding compounds taught herein, or a pharmaceutically acceptable salt thereof may be administered to the eye (e.g. by way of an ophthalmic ointment or other suitable administration routes) before the measurement. In a preferred embodiment, any of the tau-binding compounds taught herein or a pharmaceutically acceptable salt thereof is administered to the eye at least 2 hours, preferably at least 4 hours, more preferably at least 8 hours, even more preferably at least 12 hours and most preferably at least 18 hours prior to the measurement of fluorescence. In a preferred embodiment, any of the tau-binding compounds taught herein or a pharmaceutically acceptable salt thereof is administered to the eye at least 18 hours prior to fluorescence measurements, wherein virtually no unbound tau-binding compound is present in ocular tissue at the time of fluorescence measurement. The amount of tau-binding compound or a pharmaceutically acceptable salt thereof bound to tau protein in ocular tissue is determined by fluorescence measurement, preferably in the retina.

In accordance with an embodiment of the invention, an increase in binding of a fluorophore compound to an ocular tissue, e.g., the retina, compared to a normal control level of binding indicates that the mammal is suffering from or is at risk of developing AD. As used herein, a “fluorophore” or “fluorophore compound” is any substance having desirable fluorescent characteristics when illuminated with light of a certain wavelength and/or polarization property. As used herein, a “tau protein” comprises at least one of: a plurality of paired helical Tau-filaments; a neurofibrillary tangle; a Tau protein aggregate precursor; and a Tau protein aggregate. Preferably, in techniques discussed herein, the fluorophore is a “tau-binding compound,” which as used herein means a compound that binds to a tau protein, where “tau protein” is as defined above. Such a fluorophore may be a tau-binding compound that naturally fluoresces when exposed to light of a certain wavelength and/or polarization property. Alternatively or in addition, the fluorophore may be a compound that includes a fluorescent tag portion in combination with a tau-binding compound portion, where the tau-binding compound portion would generally not exhibit the desired fluorescence characteristics in the absence of the fluorescent tag. In one embodiment, the fluorophore has the following properties: exhibits good solubility in any medium in which the fluorophore is used; penetrates the retina of the eye; and binds to tau protein. The fluorophore may have different fluorescent characteristics when bound to tau and when unbound. For example, the spectral intensity and/or time decay rate of fluorescence of the fluorophore may change when the fluorophore is bound to tau as compared to when it is unbound. In an embodiment, the tau-binding compound produces a significantly greater intensity of emitted fluorescence when exposed to the excitation wavelength and bound to the tau protein, as compared with intensity of emitted fluorescence by the tau-binding compound when exposed to the excitation wavelength and unbound to the tau protein. For example, the significantly greater intensity of emitted fluorescence can comprise at least one of at least five times greater intensity of emitted fluorescence, at least ten times greater intensity of emitted fluorescence, and at least twenty times greater intensity of emitted fluorescence

As used herein, “ocular tissue” can include any tissue of an eye and/or the optic nerve of a mammal, such as a retina or lens of the eye. For example, the retina can include one or more of: an inner nuclear layer of the retina, and a retinal ganglion cell of the retina.

As used herein, “natural fluorescence” or “autofluorescence” signifies natural fluorescence in the eye that can occur independently of an introduced imaging agent.

As discussed herein, a device in accordance with an embodiment of the invention may comprise a light source. As used herein a “light source” may be any light source that can be configured to emit light to illuminate the eye with at least one of a wavelength and/or a polarization of light appropriate to produce fluorescence in at least a tau-binding compound when the tau-binding compound is bound to the tau protein, in a fashion such that at least a portion of the resulting emitted fluorescence may be detected and measured.

In accordance with an embodiment of the invention, the device may use an “optical unit,” which as used herein means any unit that can be configured to receive light including fluorescence produced as a result of the illumination of the eye and to detect at least a portion of fluorescence produced by the tau-binding compound bound to the tau protein, the determining permitting distinguishing of the presence of the tau-binding compound bound to the tau protein in the eye based on the emitted fluorescence. For example, with reference to FIG. 2, the optical unit may include one or more of the objective lens 101, translation stage 104, scanner 105, TCSPC module 102, a camera, an LED, the various lenses, apertures, beam splitters, dichroic filters, the time decay calculation module, the frame grabber module, and the scanner control module. Portions of the functionality of the optical unit may be implemented by a specially-programmed general purpose computer, or by dedicated hardware, for example for performing time decay calculations.

In accordance with an embodiment of the invention, a method can include detecting at least one of: (i) at least a portion of background autofluorescence emitted from the ocular tissue, upon illumination of the ocular tissue with the light source, and (ii) at least a portion of fluorescence emitted from the ocular tissue by a quantity of the tau-binding compound that is unbound to the tau protein. The method can further include “normalizing” the determined quantity of the tau-binding compound bound to the tau protein based on at least one of: (i) the at least a portion of the background autofluorescence emitted from the corresponding region of the ocular tissue (such as the retina) and (ii) the at least a portion of the fluorescence emitted by the quantity of the unbound tau-binding compound. As used herein, such “normalizing” can include subtracting one or more of the background autofluorescence and the unbound tau-binding compound quantities from the quantity of the tau-binding compound bound to the tau protein; and can include determining a ratio of one or more of such quantities; and can include using such a normalized result as a normalized measure of the quantity of the tau-binding compound bound to the tau protein.

In accordance with an embodiment of the invention, the detecting the at least a portion of the fluorescence, emitted from the ocular tissue by the tau-binding compound bound to the tau protein, is used for aiding in diagnosis of a disease, such as an amyloidogenic disease or disorder. The detecting can be used for aiding in diagnosis of a disease selected from the group consisting of Alzheimer's disease (AD), familial AD, Sporadic AD, Creutzfeld-Jakob disease, variant Creutzfeld-Jakob disease, spongiform encephalopathies, Prion diseases (including scrapie, bovine spongiform encephalopathy, and other veterinary prionopathies), Parkinson's disease, Huntington's disease (and trinucleotide repeat diseases), amyotrophic lateral sclerosis, Down's Syndrome (Trisomy 21), Pick's Disease (Frontotemporal Dementia), Lewy Body Disease, neurodegeneration with brain iron accumulation (Hallervorden-Spatz Disease), synucleinopathies (including Parkinson's disease, multiple system atrophy, dementia with Lewy Bodies, and others), neuronal intranuclear inclusion disease, tauopathies (including progressive supranuclear palsy, Pick's disease, corticobasal degeneration, hereditary frontotemporal dementia (with or without Parkinsonism), a pre-morbid neurodegenerative state and Guam amyotrophic lateral sclerosis/parkinsonism dementia complex).

As used herein, methods of detecting fluorescence that are used in “aiding in the diagnosis” signifies such methods which can assist in diagnosis but which do not in themselves allow for full diagnosis. For example, full diagnosis may include assessment of a patient's symptoms, behavior and other factors that lead to diagnosis of diseases such as amyloidogenic and neurodegenerative diseases and disorders, so that the methods of detecting fluorescence taught herein can be used in conjunction with other data and a full range of factors in order to assist with such a diagnosis.

In accordance with an embodiment of the invention, the above imaging techniques for detecting tau proteins in ocular tissue can be combined with another type of imaging, for example, a technique for detecting amyloid proteins, in order to aid in a diagnosis. Techniques taught herein can be combined with the use of methods, devices and compounds taught in U.S. Pat. Nos. 8,955,959, 9,220,403 and U.S. Patent Application Publication No. 2015/0118163, the entire teachings of which applications are hereby incorporated herein by reference.

### REFERENCES

(1) Leger F, Fernagut P O, Canron M H, Leoni S, Vital C, Tison F, Bezard E, Vital A. Protein aggregation in the aging retina. J Neuropathol Exp Neurol 2011; 70:63-8. [PMID: 21157377].

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of optical detection of a tau protein in ocular tissue, the method comprising:
illuminating the ocular tissue with a light source, the light source having an excitation wavelength appropriate to produce fluorescence in at least a tau-binding compound when bound to the tau protein in the ocular tissue, the tau-binding compound having been introduced to the ocular tissue and specifically binding to the tau protein; and
detecting at least a portion of fluorescence, emitted from the ocular tissue by the tau-binding compound bound to the tau protein, upon illumination of the ocular tissue with the light source, the emitted fluorescence having an emission wavelength in the range of from about 550 nm to about 1400 nm.

2. The method of claim 1, wherein the excitation wavelength is in the range of from about 550 nm to about 1400 nm.

3. The method of claim 1, wherein the emission wavelength is in the range of from about 750 nm to about 1400 nm.

4. The method of claim 1, wherein the excitation wavelength is in the range of from about 750 nm to about 1400 nm.

5. The method of claim 1, further comprising:
determining at least one of an intensity and a time decay rate of the at least a portion of fluorescence emitted from the ocular tissue by the tau-binding compound bound to the tau protein; and
determining a quantity of the tau-binding compound bound to the tau protein based on at least one of the intensity and the time decay rate.

6. The method of claim 5, further comprising:
detecting at least one of: (i) at least a portion of background autofluorescence emitted from a corresponding portion of the ocular tissue, upon illumination of the ocular tissue with the light source, and (ii) at least a portion of fluorescence emitted from the ocular tissue by a quantity of the tau-binding compound that is unbound to the tau protein; and
normalizing the determined quantity of the tau-binding compound bound to the tau protein based on at least one of: (i) the at least a portion of the background autofluorescence emitted from the corresponding portion of the ocular tissue and (ii) the at least a portion of the fluorescence emitted by the quantity of the unbound tau-binding compound.

7. The method of claim 5, further comprising performing a time-correlation single photon counting of the at least a portion of fluorescence emitted from the ocular tissue by the tau-binding compound bound to the tau protein.

8. The method of claim 1, wherein the emission wavelength differs from the excitation wavelength by a wavelength shift towards an increased wavelength.

9. The method of claim 8, wherein the wavelength shift comprises a shift of between about 10 nm and about 100 nm.

10. The method of claim 1, wherein the tau-binding compound produces a significantly greater intensity of emitted fluorescence when exposed to the excitation wavelength and bound to the tau protein, as compared with intensity of emitted fluorescence by the tau-binding compound when exposed to the excitation wavelength and unbound to the tau protein.

11. The method of claim 10, wherein the significantly greater intensity of emitted fluorescence comprises at least one of at least five times greater intensity of emitted fluorescence, at least ten times greater intensity of emitted fluorescence, and at least twenty times greater intensity of emitted fluorescence.

12. The method of claim 1, wherein the tau-binding compound comprises:
a terminal electron-rich donor group;
a terminal electron-deficient acceptor group; and
a polarizable pi-conjugated system bridging the terminal electron-rich donor group and the terminal electron-deficient acceptor group.

13. The method of claim 1, wherein the tau-binding compound is a compound with the following structure: 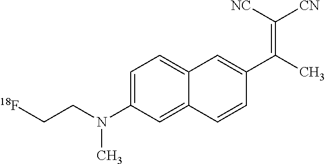 [SMILES: CC(=C(C#N)C#N)c1ccc2cc(N(C)CC[18F])ccc2c1]
or a pharmaceutically acceptable salt thereof.

14. The method of claim 1, wherein the tau-binding compound is a compound with the following structure: 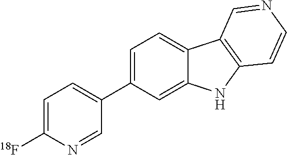 [SMILES: [18F]c1ccc(-c2ccc3c(c2)[nH]c2ccncc23)cn1]
or a pharmaceutically acceptable salt thereof.

15. The method of claim 1, wherein the tau-binding compound is a compound with the following structure: 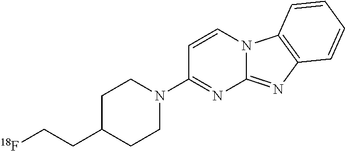 [SMILES: [18F]CCC1CCN(c2ccn3c(n2)nc2ccccc23)CC1]
or a pharmaceutically acceptable salt thereof

16. The method of claim 1, wherein the tau protein comprises at least one of: a plurality of paired helical Tau-filaments; a neurofibrillary tangle; a Tau protein aggregate precursor; and a Tau protein aggregate.

17. The method of claim 1, wherein the ocular tissue comprises at least a portion of a retina of an eye.

18. The method of claim 17, wherein the ocular tissue comprises at least one of an inner nuclear layer of the retina, and a retinal ganglion cell of the retina.

19. The method of claim 1, wherein the detecting the at least a portion of the fluorescence, emitted from the ocular tissue by the tau-binding compound bound to the tau protein, is used for aiding in diagnosis of disease.

20. The method of claim 1, wherein the detecting the at least a portion of the fluorescence, emitted from the ocular tissue by the tau-binding compound bound to the tau protein, is used for aiding in diagnosis of an amyloidogenic disease.

21. The method of claim 1, wherein the detecting the at least a portion of the fluorescence, emitted from the ocular tissue by the tau-binding compound bound to the tau protein, is used for aiding in diagnosis of a disease selected from the group consisting of Alzheimer's disease (AD), familial AD, Sporadic AD, Creutzfeld-Jakob disease, variant Creutzfeld-Jakob disease, spongiform encephalopathies, Prion diseases (including scrapie, bovine spongiform encephalopathy, and other veterinary prionopathies), Parkinson's disease, Huntington's disease (and trinucleotide repeat diseases), amyotrophic lateral sclerosis, Down's Syndrome (Trisomy 21), Pick's Disease (Frontotemporal Dementia), Lewy Body Disease, neurodegeneration with brain iron accumulation (Hallervorden-Spatz Disease), synucleinopathies (including Parkinson's disease, multiple system atrophy, dementia with Lewy Bodies, and others), neuronal intranuclear inclusion disease, tauopathies (including progressive supranuclear palsy, Pick's disease, corticobasal degeneration, hereditary frontotemporal dementia (with or without Parkinsonism), a pre-morbid neurodegenerative state and Guam amyotrophic lateral sclerosis/parkinsonism dementia complex).

22. The method of claim 1, comprising aligning the light source with at least a portion of the ocular tissue.

23. The method of claim 22, wherein aligning the light source comprises forming a camera image of a retina to be illuminated with the light source.

24. A device for optical detection of a tau protein in ocular tissue, the device comprising:
a light source configured to emit light to illuminate the ocular tissue, the light source having an excitation wavelength appropriate to produce fluorescence in at least a tau-binding compound when bound to the tau protein in the ocular tissue, the tau-binding compound having been introduced to the ocular tissue and specifically binding to the tau protein; and
an optical unit configured to detect at least a portion of fluorescence, emitted from the ocular tissue by the tau-binding compound bound to the tau protein, upon illumination of the ocular tissue with the light source, the emitted fluorescence having an emission wavelength in the range of from about 550 nm to about 1400 nm.

25. The device of claim 24, wherein the excitation wavelength is in the range of from about 550 nm to about 1400 nm.

26. The device of claim 24, wherein the emission wavelength is in the range of from about 750 nm to about 1400 nm.

27. The device of claim 24, wherein the excitation wavelength is in the range of from about 750 nm to about 1400 nm.

28. The device of claim 24, wherein the optical unit is further configured to determine at least one of an intensity and a time decay rate of the at least a portion of fluorescence emitted from the ocular tissue by the tau-binding compound bound to the tau protein; and to determine a quantity of the tau-binding compound bound to the tau protein based on at least one of the intensity and the time decay rate.

29. The device of claim 28, wherein the optical unit is further configured to:
detect at least one of: (i) at least a portion of background autofluorescence emitted from a corresponding portion of the ocular tissue, upon illumination of the ocular tissue with the light source, and (ii) at least a portion of fluorescence emitted from the ocular tissue by a quantity of the tau-binding compound that is unbound to the tau protein; and
normalize the determined quantity of the tau-binding compound bound to the tau protein based on at least one of: (i) the at least a portion of the background autofluorescence emitted from the corresponding portion of the ocular tissue and (ii) the at least a portion of the fluorescence emitted by the quantity of the unbound tau-binding compound.

30. The device of claim 24, wherein the optical unit further comprises a time decay calculation module.

31. The device of claim 28, wherein the optical unit comprises a time correlation single photon count module configured to perform a time-correlation single photon counting of the at least a portion of fluorescence emitted from the ocular tissue by the tau-binding compound bound to the tau protein.

32. The device of claim 24, wherein the emission wavelength differs from the excitation wavelength by a wavelength shift towards an increased wavelength.

33. The device of claim 32, wherein the wavelength shift comprises a shift of between about 10 nm and about 100 nm.

34. The device of claim 24, wherein the ocular tissue comprises at least a portion of a retina of an eye.

35. The device of claim 34, wherein the ocular tissue comprises at least one of an inner nuclear layer of the retina, and a retinal ganglion cell of the retina.

36. The device of claim 24, further comprising a camera configured to form a camera image of a retina to be illuminated with the light source.
